(19) United States
(12) Patent Application Publication
(10) Pub. No.: US 2025/0146932 A1
(43) Pub. Date: May 8, 2025

(54) power transformer fault detection device

(71) Applicant: Zhejiang Wanli University, Ningbo City (CN)

(72) Inventors: Danjiang Chen, Ningbo City (CN); Wen Zheng, Ningbo City (CN); Hairen Wang, Ningbo City (CN); Yutian Liu, Ningbo City (CN); Shaozhong Zhang, Ningbo City (CN)

(73) Assignee: Zhejiang Wanli University, Ningbo City (CN)

(21) Appl. No.: 19/017,717

(22) Filed: Jan. 12, 2025

(30) Foreign Application Priority Data
Nov. 11, 2024 (CN) CN202411601020.4

(51) Int. Cl. G01N 21/3577; G01N 33/28

(52) U.S. Cl. CPC G01N 21/3577; G01N 33/28; G01N 2201/0636

(57) ABSTRACT

The present application relates to the field of power equipment detection devices, and in particular to a power transformer fault detection device, which includes a detection component for detecting the oil composition of the transformer, and also includes a rotating part comprising multiple groups of oil pools that are evenly spaced around the rotating axis of the rotating part. The multiple groups of oil pools are connected to oil outlets at different heights on the transformer, and a light-transmitting window is provided on the oil pool. A detection component is installed on the rotating part, and can detect the composition of the liquid in the oil pool through the light-transmitting window. The present application has the effect of allowing staff to monitor the working status of the transformer in a timely manner and reducing the labor burden of the staff.

### TECHNICAL FIELD

The present invention relates to the technical field of power equipment detection devices, and in particular to a power transformer fault detection device

### BACKGROUND ART

Transformers are devices that use the principle of electromagnetic induction to change AC voltage. There are many types of transformers. They include oil-immersed transformers, dry-type transformers, etc. The main difference between oil-immersed transformers and ordinary dry-type transformers is that the winding core of oil-immersed transformers is immersed in transformer oil, and the transformer oil is used to insulate and dissipate heat for the core and winding.

During the use of oil-immersed transformer oil, electric arcs will cause the transformer oil to decompose, or the transformer oil will absorb moisture in the air, causing the transformer oil to exceed the standard. By detecting the changes in the composition of the transformer oil, the working state of the transformer can be reflected, so that it can be judged whether the oil-immersed transformer is stable during operation, or whether there is a risk of failure. Usually, an oil outlet is set on the oil tank of the oil-immersed transformer. When the transformer oil needs to be tested, the staff will release the oil inside the transformer into a test tube through the oil outlet, and then transport it to the laboratory for testing. The transformer oil is usually tested using an infrared detector, also known as Raman spectroscopy.

Some large transformers have oil tanks that are two meters or even higher. The temperature and sedimentation of the transformer oil at different heights inside are different. Therefore, large transformers are usually equipped with multiple oil outlets at their own height. During the inspection process, oil discharge inspections need to be performed from multiple oil outlets. This process is relatively cumbersome and consumes a lot of manpower, which affects the transformer inspection cycle. The transformer oil inspection cycle is generally once every three years. This method cannot detect changes in the transformer oil in a timely manner, and therefore cannot timely know the working status of the transformer.

### SUMMARY

In order to reduce the timeliness of the staff's detection of the transformer's operating status and reduce the staff's labor burden, the present application provides a power transformer fault detection device.

The power transformer fault detection device provided by the present application adopts the following technical scheme:

A power transformer fault detection device includes a detection component for detecting the transformer oil composition, and also includes a rotating member that can rotate and multiple groups of oil pools that are evenly spaced around the rotating axis of the rotating member. The multiple groups of oil pools are connected to the oil outlet nozzles at different heights on the transformer, and a light-transmitting window is provided on the oil pool. The detection component is installed on the rotating member and can detect the composition of the liquid in the oil pool through the light-transmitting window.

By adopting the above technical scheme, the oil pool is connected to the oil outlet nozzle of the transformer oil tank. After the transformer oil in the transformer oil tank is pumped into the oil pool by the oil pump, the detection component then monitors the transformer oil in the oil pool in real time through the light-transmitting window, which is convenient for timely detection of faults during the operation of the transformer, and at the same time reduces the labor burden of the staff. Moreover, the detection component can be driven to rotate by the rotating member, so as to detect the transformer oil in the multiple groups of oil pools, thereby improving the accuracy of transformer oil detection.

Optionally, the detection assembly includes an infrared generator for emitting infrared rays and a detector for detecting infrared rays, and also includes a reflector arranged on the side of the oil pool away from the rotating member, and the reflector is provided with a reflective surface capable of reflecting light, and the reflective surface can guide the light to extend toward the detector.

By adopting the above technical solution, during the detection process, the infrared generator emits infrared light, and the infrared light passes through the transformer oil in the oil pool through the light-transmitting window and then passes out from the other side of the oil pool. Then it extends to the reflector, and under the action of the reflector, the light is guided away from the reflection of the light to hit the detector, the detector detects the light, and then determines the composition of the transformer oil according to the known database to realize the detection of the transformer oil. By setting the reflector to reflect the light and guide the light propagation, the detector and the infrared generator can be set on the rotating member at the same time, which reduces the production cost of the equipment.

Optionally, it also includes a protective cover, which is arranged outside the oil pool, and the rotating member is rotatably connected to the inner wall of the protective cover.

By adopting the above technical solution, the protective cover can reduce the corrosion of the oil pool caused by external weather in the outdoor environment, and provide space for the fixed installation of the oil pool and the rotating part.

Optionally, each group of the oil pools includes at least two oil pools, and the multiple oil pools are interconnected. The infrared generator is movably connected to the rotating part, and the movement of the infrared generator can make the infrared rays emitted by it pass through multiple oil pools at the same time or pass through any oil pool alone.

By adopting the above technical solution, the movable infrared generator detects the transformer oil in different oil pools, reduces the situation where the detection results have large deviations due to the equipment problems of the oil pool itself, and improves the accuracy of the detection.

Optionally, the multiple oil pools in the same group are arranged in sequence along a direction perpendicular to the rotation axis of the rotating part, and the multiple oil pools in the same group are arranged in sequence along a direction perpendicular to the rotation axis of the rotating part. The two parallel sides of the oil pool are each provided with a light-transmitting window, and a detection channel for light to pass through is formed between the light-transmitting windows on the two sides. The detection channels are arranged in multiples, and the detection channels include a data channel and a blank channel. The blank channel is at least one, the data channel is filled with transformer oil, and the blank channel is not filled with transformer oil. The blank channels on the two oil pools are arranged in an interlaced manner.

By adopting the above technical solution, the data channel and the blank channel on the two oil pools cooperate with each other to realize the detection of transformer oil in any single oil pool, or the synchronous detection of transformer oil in multiple oil pools. Specifically, the infrared light passes through the blank channel of one of the oil pools and the data channel of another oil pool to realize the separate detection of transformer oil in one oil pool; or the infrared light passes through the data channels of multiple oil pools to realize the synchronous detection of transformer oil in multiple oil pools.

Optionally, multiple detection channels are arranged along a direction parallel to the rotation axis of the rotating member, and the infrared generator is rotatably connected to the rotating member at a position away from the oil pool. Rotating the infrared generator can cause the end of the infrared generator to be displaced in a direction parallel to the rotation axis of the rotating member.

By adopting the above technical solution, multiple detection channels are arranged along the rotation axis of the rotating member. When the infrared generator is rotated, the transmitting end of the infrared generator moves in a direction parallel to the rotation axis of the rotating member, thereby making the end of the infrared generator correspond to different detection channels.

Optionally, a sliding component and a rotating component are provided on the reflector, the sliding component is used to drive the reflector to slide along a direction parallel to the rotation axis of the rotating member, the rotating component is used to drive the reflector to rotate, and the reflective surface is set to multiple around the rotation axis of the reflector.

By adopting the above technical solution, sliding the reflector can make the reflector correspond to different detection channels, and rotating the reflector can make different reflective surfaces correspond to the detection channels, thereby guiding infrared light in various states.

Optionally, the sliding assembly includes a sliding block slidably connected to the protective cover, the rotating assembly includes a gear rotatably connected to the sliding block and connected to the reflector, and also includes a rack fixed to the protective cover, and the rack is provided with a plurality of corresponding detection channels, and the sliding of the sliding block can drive the gear to mesh with the rack.

By adopting the above technical solution, the sliding sliding block can drive the reflector to move so that it corresponds to different detection channels. At the same time, during the sliding process of the sliding block, the gear meshes with the rack, driving the reflector to rotate, and synchronously realizing the adjustment of the position of the reflective surface.

Optionally, the detection assembly further includes a transition piece, which is arranged on one side of the reflector in a direction parallel to the rotation axis of the rotating member, and a reflective surface is arranged on the transition piece, and the reflective surface on the transition piece is used to guide the light irradiated on the reflective surface to extend in a direction perpendicular to the rotation axis of the rotating member to a direction close to the detector, and the reflective surface on the reflector can guide the light to extend in a direction parallel to the rotation axis of the rotating member to a direction close to the transition piece.

By adopting the above technical solution, multiple detection channels are arranged along the rotation axis of the rotating member, and only when the reflector guides the light to extend in a direction close to the detector, there is a situation where the infrared light interferes with the oil pool. By reflecting the infrared light from one side of the oil pool by the transition piece, on the one hand, the interference between the infrared light and the oil pool is reduced, and on the other hand, the extension of the infrared light is made more regular.

Optionally, the protective cover is also provided with an adjustment component, which is used to drive the infrared generator to rotate. The adjustment component includes an adjustment rod fixed on the protective cover and a connecting piece connected to the infrared generator. The adjustment rod is provided with an adjustment slot, and the end of the connecting piece away from the infrared generator is inserted into the adjustment slot. The rotation of the rotating piece can drive the infrared generator to rotate around its own rotation axis.

By adopting the above technical solution, during the rotation of the rotating piece, the infrared generator is driven to rotate around its own rotation axis, and multiple groups of oil pools are detected in sequence. During the rotation of the rotating piece, the infrared emitter is driven to rotate around its own axis, so that the infrared generator can correspond to different detection channels, and a comprehensive detection of multiple detection channels of multiple groups of oil pools is achieved.

### FIGURES

FIG. 1 is a schematic diagram of the overall structure of the embodiment of the present application;

FIG. 2 is a schematic diagram of the internal structure of the embodiment of the present application at a first angle;

FIG. 3 is a schematic diagram of the internal structure of the embodiment of the present application at a second angle;

FIG. 4 is a schematic diagram of the structure of the rotating assembly of the embodiment of the present application;

FIG. 5 is a schematic diagram of the structure of the sliding assembly of the embodiment of the present application;

FIG. 6 is an enlarged view of part A in FIG. 5 of the embodiment of the present application;

FIG. 7 is a schematic diagram of the structure of the internal structure of the embodiment of the present application at a third angle (mainly used to show the position of the adjustment slot).

Figure numerals: 1. oil pool; 11. light-transmitting window; 12. oil inlet; 13. oil outlet; 14. hard oil pipeline; 2. detection component; 21. infrared generator; 22. detector; 23. reflector; 231. reflective surface; 24. transition piece; 3. protective cover; 31. rotating part; 4. detection channel; 41. data channel; 42. blank channel; 43. blocking cylinder; 5. sliding component; 51. sliding block; 52. mounting bracket; 53. sliding groove; 54. driving rod; 55. connecting rod; 6. rotating component; 61. gear; 62. driving bar; 7. adjusting component; 71. adjusting rod; 72. connecting piece; 721. connecting rod; 722. connecting ball; 73. adjusting groove; 731. circular portion; 732. guiding portion.

### DETAILED DESCRIPTION

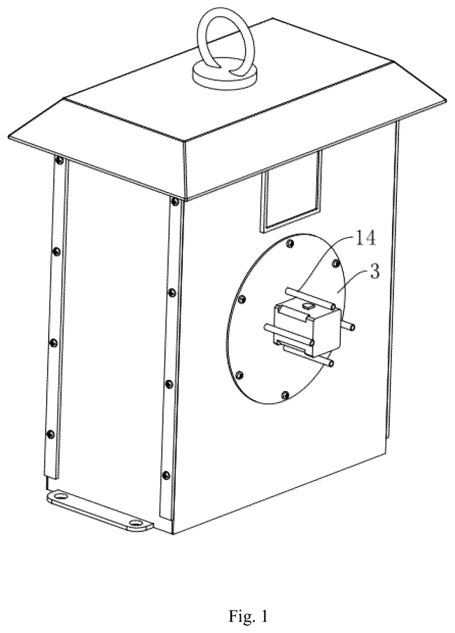

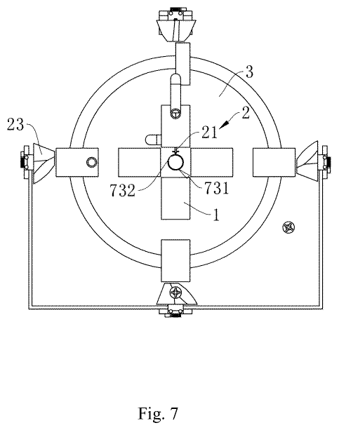

The present application is further described in detail below in conjunction with FIGS. 1-7.

The embodiment of the present application discloses a power transformer fault detection device.

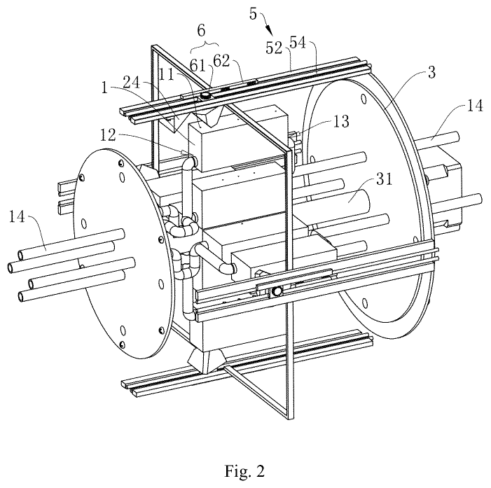

Referring to FIGS. 1 and 2, a power transformer fault detection device includes an oil pool 1 and a detection component 2. The oil pool 1 is connected to the oil outlet of the transformer oil tank. The transformer oil inside the transformer oil tank can be discharged into the oil pool 1. The oil pool 1 is provided with a light-transmitting window 11. The detection component 2 can detect the transformer oil in the oil pool 1 through the light-transmitting window 11. During the use of the fault detection device, the oil pool 1 is connected to the inside of the transformer oil tank, and the transformer oil is transported to the oil pool 1 through a liquid pump, so that the transformer oil can be monitored in real time, which is convenient for the staff to promptly discover the faults during the operation of the transformer, and at the same time reduces the labor burden of the staff.

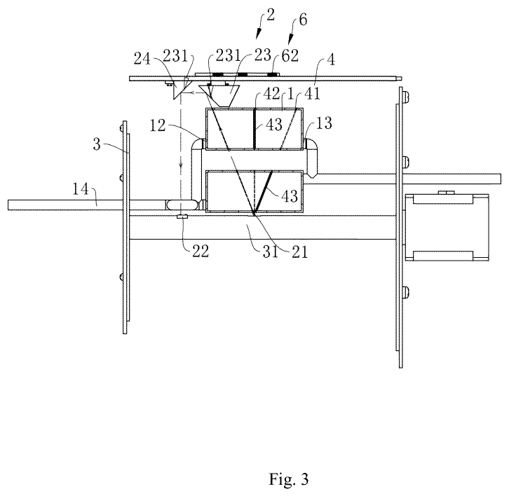

Referring to FIGS. 2 and 3, the oil pool 1 is a rectangular block structure as a whole, and is hollow inside. An oil inlet 12 and an oil outlet 13 are respectively provided on its two parallel sides, and a hard oil pipe 14 is provided at the oil outlet 13 and the oil inlet 12. In addition to the oil outlet, the transformer is also provided with an oil inlet, and the ends of the two hard oil pipes 14 away from the oil pool 1 are respectively connected to the oil inlet and the oil outlet on the transformer. The circulation from the transformer to the oil pool 1 is realized by oil pump delivery, which can ensure the liquid level of the transformer oil inside the transformer tank. Specifically, the light-transmitting window 11 is formed in the following manner: a through hole connected to the inside of the oil pool 1 is opened, and a light-transmitting sheet (which can be a glass sheet) is provided at the through hole to form a light-transmitting window 11, so as to facilitate the detection of the transformer oil inside the oil pool 1.

Referring to FIGS. 1 and 2, the outer cover of the oil pool 1 is provided with a protective cover 3 to reduce the occurrence of the external environment affecting the service life of the oil pool 1. A rotating member 31 is arranged inside the protective cover 3. The rotating member 31 is a cylindrical structure as a whole. It is arranged horizontally and rotates around its own axis to be connected to the inner wall of the protective cover 3. Multiple groups of oil pools 1 are evenly spaced around the rotation axis of the rotating member 31. The oil outlet nozzles at different heights on the large transformer are connected to different oil pools 1. The detection component 2 is installed on the rotating member 31. Rotating the rotating member 31 can drive the detection component 2 to rotate, so as to detect the transformer oil in different oil pools 1, realize the detection of transformer oil at different heights in the transformer, and improve the accuracy of the detection.

Referring to FIGS. 2 and 3, the detection component 2 includes an infrared generator 21 and a detector 22. The infrared generator 21 is used to emit infrared rays, and the detector 22 is used to measure infrared rays. The detection component 2 also includes a reflector 23. The reflector 23 is arranged on the side of the liquid pool away from the rotating member 31, and a reflective surface 231 is arranged on the reflector 23. The reflective surface 231 is used to change the propagation path of the infrared light. During the detection process, the infrared generator 21 emits infrared light toward the oil pool 1, and the infrared light passes through the transformer oil in the oil pool 1 and extends to the reflective surface 231. Under the action of the reflective surface 231, the infrared light extends toward the detector 22. By setting the reflector 23, the number of detectors 22 is reduced, and the production cost of the equipment is reduced. After the detector 22 detects the infrared light, the processor performs signal processing to obtain the composition of the transformer oil and realize the detection of the transformer oil.

Referring to FIGS. 2 and 3, the detection component 2 also includes a transition piece 24, which is fixed on the protective cover 3 and is arranged on one side of the reflector 23 in a direction parallel to the rotation axis of the rotating member 31. The transition piece 24 is also provided with a reflective surface 231 that can reflect light. In this embodiment, the angle between the reflective surface 231 on the reflector 23 and the rotation axis of the reflector 23 is determined according to the principle of light reflection (the reflected light and the incident light and the normal are on the same plane; the reflected light and the incident light are separated on both sides of the normal; the reflection angle is equal to the incident angle), so that the infrared light passes through the detection channel 4 and is covered on the reflective surface 231 of the reflector 23 and then extends in the direction parallel to the rotation axis of the rotating member 31 toward the direction close to the transition member 24. The reflective surface 231 on the transition member 24 is 45 degrees to the direction parallel to the rotation axis of the rotating member 31, so that the infrared light extends in the direction perpendicular to the rotation axis of the rotating member 31 toward the direction close to the rotating member 31.

Referring to FIGS. 2 and 3, each group of oil pools 1 includes at least two oil pools 1, and the infrared generator 21 is movably connected to the rotating frame. The position change of the infrared generator 21 can make the infrared light pass through different numbers or different oil pools 1. During the detection process, the oil pools 1 in the same group of oil pools 1 are detected separately, or multiple oil pools 1 are detected at the same time, so as to reduce the situation where the detection result is large due to the equipment reasons of the oil pool 1 itself, and improve the detection accuracy.

Referring to FIGS. 2 and 3, in this embodiment, each group of oil pools 1 includes two oil pools 1, and the two oil pools 1 are connected in parallel, that is, the oil inlets 12 of the two oil pools 1 are connected to each other, and the oil outlets 13 of the two oil pools 1 are connected to each other.

Referring to FIGS. 2 and 3, light-transmitting windows 11 are provided on two mutually parallel sides of the oil pool 1, and a detection channel 4 is formed between the two light-transmitting windows 11. In this embodiment, the light-transmitting windows 11 are circular, and the detection channel 4 forms a cylindrical channel correspondingly. The detection channels 4 on the two oil pools 1 correspond to each other, and the light-transmitting windows 11 of the corresponding detection channels 4 on the two oil pools 1 are linearly arranged (that is, the corresponding detection channels 4 are coaxial), so that the infrared light can pass through the two oil pools 1 at the same time.

Referring to FIGS. 2 and 3, the detection channel 4 includes at least one blank channel 42 and at least one data channel 41. In this embodiment, one blank channel 42 is set. The blank channel 42 is not filled with transformer oil, and the blank channel 42 can be in a vacuum state or in a gas-filled state. A blocking tube 43 is provided between the two light-transmitting windows 11 corresponding to the blank channel 42. The blocking tube 43 is a cylindrical structure, which is located inside the oil pool 1, and the two ends are integrally formed and connected to the two mutually parallel inner walls of the oil pool 1, and the inner wall of the blocking tube 43 is flush with the inner wall of the light-transmitting window 11, forming a blank channel 42 that runs through the entire oil pool 1.

Referring to FIGS. 2 and 3, the blank channels 42 on the two oil pools 1 are arranged at intervals. During the detection process, the active infrared generator 21 can make the infrared light pass through different detection channels 4. There are many situations in which the infrared light passes through the oil pool 1, as follows. For the convenience of description, the two oil pools 1 in the same group are defined as the first detection pool and the second detection pool. The infrared ray passes through the blank channel 42 on the first detection pool and the detection channel 4 on the second detection pool, the detection channel 4 on the first detection pool and the blank channel 42 on the second detection pool, and the detection channel 4 on the first detection pool and the second detection pool, so as to realize the separate detection of multiple oil pools 1 and the joint detection of multiple oil pools 1, and reduce the occurrence of detection errors caused by the equipment of the oil pool 1 itself.

Referring to FIGS. 2 and 3, the infrared generator 21 is rotatably connected to the rotating member 31 at a position away from the oil pool 1, and the rotation axis of the infrared generator 21 is perpendicular to the rotation axis of the rotating member 31. Rotating the infrared generator 21 can make the head of the infrared generator 21 (which can also be understood as the end that emits infrared light, the emitting end) move in a direction parallel to the rotation axis of the rotating member 31. This allows the head of the infrared generator 21 to correspond to different detection channels 4 during the rotation process, so as to realize multiple detections of the oil pool 1.

Referring to FIGS. 2 and 3, the reflector 23 is rotatably arranged on the protective cover 3 and can move in a direction parallel to the rotation axis of the rotating member 31, so that the reflector 23 corresponds to different detection channels 4, which is convenient for reflecting infrared light to the detector 22. The rotation axis of the reflector 23 is perpendicular to the rotation axis of the rotating member 31 and perpendicular to the rotation axis of the infrared generator 21. There are multiple reflective surfaces 231 on the reflector 23. Multiple reflective surfaces 231 are arranged around the rotation axis of the reflector 23, and the multiple reflective surfaces 231 have different angles with the rotation axis of the reflector 23, so as to reflect infrared light passing through different detection channels 4 to the detector 22.

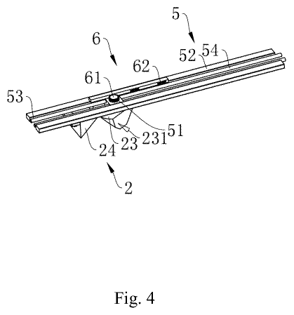

Referring to FIGS. 2 and 4, a sliding assembly 5 is arranged on the side of the reflector 23 away from the rotating member 31, and the sliding assembly 5 includes a sliding block 51 and a mounting frame 52, the mounting frame 52 is welded to the protective cover 3, and a sliding groove 53 with a length direction parallel to the rotation axis of the rotating member 31 is opened on the mounting frame 52, and the sliding block 51 is slidably connected in the sliding groove 53. The reflector 23 is rotatably connected to the sliding block 51 to realize the sliding connection between the reflector 23 and the protective cover 3. The protective cover 3 is also provided with a driving rod 54 for driving the sliding block 51 to move. The driving rod 54 is arranged along the sliding direction of the sliding block 51 and is rotatably connected to the protective cover 3. The driving rod 54 is threadedly connected to the sliding block 51, and the rotation of the driving rod 54 can drive the sliding block 51 to move to drive the sliding block 51. In this embodiment, the sliding block 51 is driven by a servo motor.

Referring to FIGS. 2 and 4, the sliding block 51 is provided with a rotating assembly 6 for driving the reflector 23 to rotate. The rotating assembly 6 includes a gear 61 and a driving bar 62. The gear 61 is rotatably arranged on the side of the sliding block 51 away from the rotating member 31 and is fixedly connected to the reflector 23. The rotating gear 61 can drive the reflector 23 to rotate. The driving bar 62 is a rectangular strip structure as a whole. The driving bar 62 is arranged along the axial direction of the rotating member 31 and fixed on the mounting frame 52.

Referring to FIGS. 2 and 4, the driving bar 62 includes two multiple racks arranged in a direction parallel to the rotation axis of the rotating member 31. In the initial state of the detection device in this embodiment, the reflector 23 is located at the position of the detection channel 4 close to the transition member 24. No rack is set at this position, and racks are set at the positions of the other detection channels 4.

Referring to FIGS. 2 and 4, the rack is located on the side of the detection channel 4 close to the transition member 24. Slide the sliding block 51, and before the reflector 23 reaches the position of the adjacent detection channel 4, the gear 61 can approach the rack and mesh with the rack. As the sliding block 51 slides, the reflector 23 is driven to rotate, and the position of the reflective surface 231 is adjusted, and the position of the reflector 23 as a whole is adjusted. The generatrix circumference of the gear 61 is defined as L, and the length of the rack is defined as L/3. When the reflector 23 moves to the specified position, the rack is disengaged from the gear 61, and the reflector 23 rotates 120° at this time, so that the adjacent reflective surface 231 acts on the infrared light.

Referring to FIGS. 2 and 4, the sliding assembly also includes a linkage rod 55, which is arranged between two adjacent sliding blocks 51. The two ends of the linkage rod 55 are respectively welded to the two sliding blocks 51 to achieve mutual connection between multiple sliding blocks 51, so that multiple sliding blocks 51 can move synchronously, which is convenient for the staff to synchronously adjust the positions of multiple sliding blocks 51.

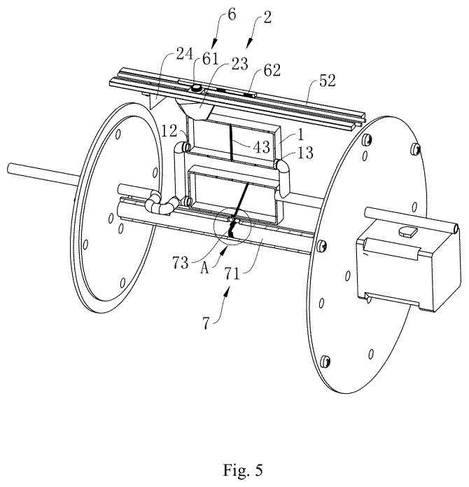

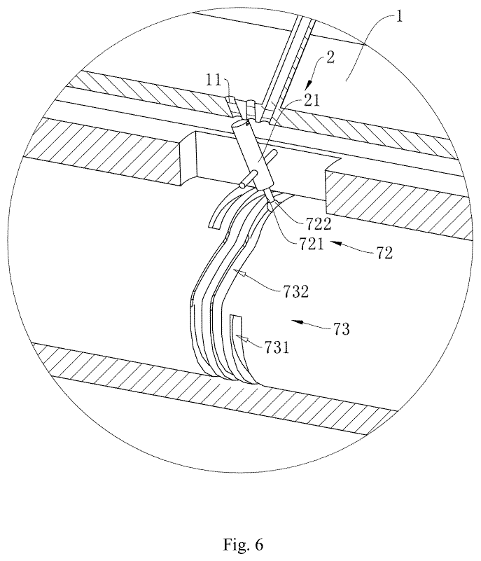

Referring to FIGS. 5 and 6, an adjustment assembly 7 for driving the infrared transmitter to rotate is arranged inside the rotating member 31, and the adjustment assembly 7 includes an adjustment rod 71 and a connecting member 72. The adjustment rod 71 is a cylindrical rod-shaped structure, and the two ends of the adjustment rod 71 are welded to the protective cover 3. A through hole is opened on the rotating member 31 corresponding to the adjustment rod 71, and the rotating member 31 is rotatably sleeved on the adjustment rod 71 to achieve a rotational connection between the rotating member 31 and the protective cover 3.

Referring to FIGS. 5 and 6, an adjustment groove 73 is provided on the adjustment rod 71, and the adjustment groove 73 includes a circumferential portion 731 and a guide portion 732. The length direction of the circumferential portion 731 extends along the circumferential direction of the adjustment rod 71, and the arc of the circumferential portion 731 is greater than 3/2π and less than 2π. The circumferential portion 731 is arranged to be multiple at intervals along the length direction of the adjustment rod 71, and in this embodiment, the circumferential portion 731 is arranged to be three. The guide portion 732 is arranged to be two corresponding to the gap between two adjacent circumferential portions 731. The two ends of the circumferential portion 731 are defined as the head end and the tail end, respectively, and the two ends of the guide portion 732 are connected to the head end and the tail end of the two adjacent circumferential portions 731, respectively. The connecting member 72 includes a connecting rod 721 and a connecting ball 722, and the connecting ball 722 is located in the adjustment groove 73 and can move along the length direction of the adjustment groove 73. One end of the connecting rod 721 is welded to the connecting ball 722, and the other end is slidably connected to the infrared generator 21 along its own length direction.

Referring to FIGS. 5 and 6, in the initial state, the connecting ball 722 is located at one end of the adjustment slot 73. The rotating member 31 is rotated, and the rotating member 31 drives the infrared generator 21 to move, and at the same time drives the connecting ball 722 to move in the circumference 731 along the length direction of the circumference 731. When the connecting ball 722 is located in the guide portion 732, the connecting ball 722 is driven to displace in a direction parallel to the rotation axis of the rotating member 31, thereby driving the infrared generator 21 to rotate a certain angle. When the connecting ball 722 enters the adjacent circumference 731, it continues to move along the length direction of the circumference 731, and so on, completing multiple tests of the transformer oil in the oil pool 1. The guide portion 732 is set corresponding to the gap between the two adjacent groups of oil pools 1, so that the infrared generator 21 can pass through multiple groups of oil pools 1 in sequence during the movement of the connecting ball 722 in the circumference 731.

The implementation principle of a power transformer fault detection device in the embodiment of the present application is as follows: the oil pool 1 is connected to the inside of the transformer oil tank. During the transformer oil detection process, the transformer oil can be circulated between the oil pool 1 and the transformer oil tank through a liquid pump. At the same time, the detection component 2 performs a circulation detection on the transformer oil in the oil pool 1 to achieve real-time monitoring of the transformer working state, which is convenient for timely detection of faults during the operation of the transformer and reduces the labor burden of the staff.

The above are all preferred embodiments of the present application, and the protection scope of the present application is not limited thereto. Therefore, all equivalent changes made according to the structure, shape, and principle of the present application should be covered within the protection scope of the present application.

1. A power transformer fault detection device, comprising a detection component (2) for detecting transformer oil composition, characterized in that it also comprises a rotatable rotating member (31) and a plurality of oil pools (1) evenly spaced around the rotating axis of the rotating member (31), wherein the plurality of oil pools (1) are connected to oil outlets at different heights on the transformer, and the oil pools (1) are provided with a light-transmitting window (11), wherein the detection component (2) is mounted on the rotating member (31) and is capable of detecting the composition of the liquid in the oil pool (1) through the light-transmitting window (11).

2. A power transformer fault detection device according to claim 1, characterized in that the detection component (2) comprises an infrared generator (21) for emitting infrared rays and a detector (22) for detecting infrared rays, and further comprises a reflector (23) disposed on a side of the oil pool (1) away from the rotating member (31), wherein the reflector (23) is provided with a reflective surface (231) capable of reflecting light, and wherein the reflective surface (231) is capable of guiding the light to extend toward the detector (22).

3. A power transformer fault detection device according to claim 2, characterized in that: it also includes a protective cover (3), wherein the protective cover (3) is arranged outside the oil pool (1), and the rotating member (31) is rotatably connected to the inner wall of the protective cover (3).

4. A power transformer fault detection device according to claim 3, characterized in that: each group of the oil pools (1) includes at least two oil pools (1), and the multiple oil pools (1) are interconnected, wherein the infrared generator (21) is movably connected to the rotating member (31), and the movement of the infrared generator (21) can make the infrared rays emitted by it pass through multiple oil pools (1) at the same time or pass through any one oil pool (1) alone.

5. A power transformer fault detection device according to claim 4, characterized in that:
multiple oil pools (1) in the same group of the oil pools (1) are arranged in sequence along a direction perpendicular to the rotation axis of the rotating member (31), and two mutually parallel side surfaces of the oil pool (1) are each provided with a light-transmitting window (11), wherein a detection channel (4) for light to pass through is formed between the light-transmitting windows (11) on the two side surfaces, and the detection channels (4) are arranged in plurality, and wherein the detection channels (4) include a data channel (41) and a blank channel (42), and at least one blank channel (42) is arranged such that the data channel (41) is filled with transformer oil, and the blank channel (42) is not filled with transformer oil, and the blank channels (42) on the two oil pools (1) are arranged in an alternating manner.

6. A power transformer fault detection device according to claim 5, characterized in that: a plurality of the detection channels (4) are arranged in a direction parallel to the rotation axis of the rotating member (31); wherein the infrared generator (21) is rotatably connected to the rotating member (31) at a position away from the oil pool (1); and wherein the rotation of the infrared generator (21) can cause the end of the infrared generator (21) to be displaced in a direction parallel to the rotation axis of the rotating member (31).

7. A power transformer fault detection device according to claim 6, characterized in that: a sliding component (5) and a rotating component (6) are arranged on the reflector (23); the sliding component (5) is used to drive the reflector (23) to slide in a direction parallel to the rotation axis of the rotating member (31); the rotating component (6) is used to drive the reflector (23) to rotate; and the reflective surface (231) is arranged in a plurality around the rotation axis of the reflector (23).

8. A power transformer fault detection device according to claim 7, characterized in that: the sliding assembly (5) includes a sliding block (51) slidably connected to the protective cover (3), wherein the rotating assembly (6) includes a gear (61) rotatably connected to the sliding block (51) and connected to the reflector (23), and also includes a rack fixed to the protective cover (3), wherein a plurality of racks are provided corresponding to the detection channel (4), and the sliding block (51) can drive the gear (61) to mesh with the rack when sliding.

9. A power transformer fault detection device according to claim 3, characterized in that: the detection component (2) further comprises a transition piece (24), wherein the transition piece (24) is arranged on one side of the reflective piece (23) in a direction parallel to the rotation axis of the rotating piece (31), wherein the transition piece (24) is provided with a reflective surface (231), wherein the reflective surface (231) on the transition piece (24) is used to guide the light irradiated on the reflective surface (231) to extend in a direction perpendicular to the rotation axis of the rotating piece (31) toward the detector (22), and wherein the reflective surface (231) on the reflective piece (23) can guide the light to extend in a direction parallel to the rotation axis of the rotating piece (31) toward the transition piece (24).

10. A power transformer fault detection device according to claim 6, characterized in that: the protective cover (3) is also provided with an adjustment component (7), wherein the adjustment component (7) is used to drive the infrared generator (21) to rotate, wherein the adjustment component (7) comprises an adjustment rod (71) fixed on the protective cover (3) and wherein a connecting member (72) connected to the infrared generator (21), the adjustment rod (71) is provided with an adjustment groove (73), wherein one end of the connecting member (72) away from the infrared generator (21) is inserted into the adjustment groove (73), and the rotation of the rotating member (31) can drive the infrared generator (21) to rotate around its own rotation axis.
